(19) United States
(12) Patent Application Publication
(10) Pub. No.: US 2021/0186995 A1
(43) Pub. Date: Jun. 24, 2021

(54) PLASMALOGEN-CONTAINING COMPOSITION

(71) Applicant: MARUDAI FOOD CO., LTD., Osaka (JP)

(72) Inventors: Jun KAWAMURA, Osaka (JP); Hitomi KIMURA, Osaka (JP); Rihoko NAKASE, Osaka (JP); Satoshi KOTOURA, Osaka (JP)

(73) Assignee: MARUDAI FOOD CO., LTD., Osaka (JP)

(21) Appl. No.: 17/252,481

(22) PCT Filed: Jul. 4, 2019
(86) PCT No.: PCT/JP2019/026550

§ 371 Date: Dec. 15, 2020

(30) Foreign Application Priority Data
Jul. 6, 2018 (JP) 2018-129133

(51) Int. Cl. A61K 31/685; A61K 47/69; A61K 9/14; A61K 31/724

(52) U.S. Cl. CPC A61K 31/685; A61K 31/724; A61K 9/14; A61K 47/69

(57) ABSTRACT

This invention provides a technique that is capable of stably storing plasmalogen for a long period of time. More specifically, the invention provides a plasmalogen-containing solid composition comprising plasmalogen, γ-cyclodextrin, and a pH alkali adjusting agent, and having a pH of 6 to 8 when formed into a 1 mass % aqueous suspension.

### TECHNICAL FIELD

The present invention relates to a plasmalogen-containing composition, a method for producing the composition, etc. The entire contents of the documents disclosed in the present specification are incorporated herein by reference.

### BACKGROUND ART

Plasmalogen is a type of ether glycerophospholipid containing a vinyl ether bond at position 1 of the glycerol skeleton. Plasmalogens are widely distributed in animals in general, and in certain anaerobic microorganisms. In humans, plasmalogens are known to be abundant in the nervous, cardiovascular, and immune systems; etc. Plasmalogens are also known to be present in cell nuclei and synaptic clefts, suggesting that plasmalogens function widely in neural activity.

The functions of plasmalogens, such as cerebral nerve cell neogenesis action (Patent Literature (PTL) 1), central nervous system anti-inflammatory action (PTL 2), and learning and memory ability enhancing action in healthy mammals (PTL 3), have been clarified.

### CITATION LIST

### Patent Literature

PTL 1: WO2011/083827

PTL 2: WO2012/039472

PTL 3: JP2016-210696A

PTL 4: WO2017/187540

### Non-Patent Literature

NPL 1: Animal Science Journal, 85 (2), 153-161, 2014

### SUMMARY OF INVENTION

### Technical Problem

As described above, it has become clearer that plasmalogens have many advantageous effects, and the use thereof is expected to expand. On the other hand, the structure specific to plasmalogens, i.e., the vinyl ether bond, is highly reactive with active oxygen and radicals, compared to other glycerophospholipids, and plasmalogens are actually easily oxidized. Accordingly, plasmalogens have poor stability over time, and storing plasmalogens stably for a long period of time is difficult.

Therefore, the present inventors conducted research to develop a technique that is capable of stably storing plasmalogen for a long period of time.

### Solution to Problem

The present inventors found the possibility of stably maintaining plasmalogen for a long period of time by adding a pH alkali adjusting agent and γ-cyclodextrin to plasmalogen. The inventors made further improvements to accomplish the present invention.

For example, the present invention encompasses the subject matter described in the following items.

Item 1. A plasmalogen-containing solid composition comprising
plasmalogen,
γ-cyclodextrin, and
a pH alkali adjusting agent, and
having a pH of 6 to 8 when formed into a 1 mass % aqueous suspension.

Item 2. A plasmalogen-containing solid composition comprising
plasmalogen,
γ-cyclodextrin, and
at least one member selected from the group consisting of sodium citrate, sodium carbonate, sodium hydrogen carbonate, and sodium hydrogen phosphate, and
having a pH of 6 to 8 when formed into a 1 mass % aqueous suspension.

Item 3. The composition according to Item 1 or 2, which is a dry composition.

Item 4. The composition according to any one of Items 1 to 3, which is a powder composition.

Item 5. The composition according to any one of Items 1 to 4, wherein the composition comprises plasmalogen in an amount of 0.1 to 10 mass %.

Item 6. A suspension comprising
plasmalogen,
γ-cyclodextrin, and
a pH alkali adjusting agent, and
having a pH of 6 to 8.

Item 7. A suspension comprising
plasmalogen,
γ-cyclodextrin, and
at least one member selected from the group consisting of sodium citrate, sodium carbonate, sodium hydrogen carbonate, and sodium hydrogen phosphate, and
having a pH of 6 to 8.

Item 8. The suspension according to Item 6 or 7, wherein the solvent is water.

Item 9. A method for producing a plasmalogen-containing composition, comprising
step (A) of mixing at least plasmalogen, γ-cyclodextrin, a pH adjusting agent, and water to prepare a suspension having a pH of 6 to 8.

Item 10. The method according to Item 9, further comprising
step (B) of drying the suspension obtained in step (A) to obtain a dry composition.

Item 11. A method for improving the stability of plasmalogen, comprising
step (A) of mixing at least plasmalogen, γ-cyclodextrin, a pH adjusting agent, and water to prepare a suspension having a pH of 6 to 8.

Item 12. The method according to Item 11, further comprising
step (B) of drying the suspension obtained in step (A) to obtain a dry composition.

### Advantageous Effects of Invention

Provided is a technique that is capable of stably storing plasmalogen for a long period of time.

When plasmalogen is purified, the viscosity increases, making the handling thereof extremely difficult. However, the addition of a pH alkali adjusting agent and γ-cyclodextrin to plasmalogen, followed by drying, can give a solid composition (preferably a dry composition, more preferably a powder composition), thus also providing a composition in which plasmalogen can be stably stored for a long period of time, and in which handling difficulties due to high viscosity etc. are reduced. Additionally, a method for producing the composition etc. is also provided.

### BRIEF DESCRIPTION OF DRAWINGS

FIG. 1 is photographs showing the results obtained by mixing an ethanol extract concentrate of bird breast meat with an aqueous ethanol solution, and allowing the liquid mixture to stand and separate.

FIG. 2 shows the results of TLC analysis of each separated layer of FIG. 1.

FIG. 3 is a graph showing the stability over time of plasmalogen when a powder was prepared by combining plasmalogen and a cyclodextrin (α-cyclodextrin or γ-cyclodextrin).

FIG. 4 is a graph showing the stability over time of plasmalogen when a powder was prepared by a freeze-drying method by combining plasmalogen, γ-cyclodextrin, and sodium citrate.

FIG. 5 is a graph showing the stability over time of plasmalogen when a powder was prepared by a spray-drying method by combining plasmalogen, γ-cyclodextrin, and sodium citrate.

FIG. 6 is a graph showing the stability over time of plasmalogen when a powder was prepared by a freeze-drying method by combining plasmalogen, γ-cyclodextrin, and various pH alkali adjusting agents.

### DESCRIPTION OF EMBODIMENTS

Below, each embodiment encompassed by the present invention is described in more detail. The present invention preferably encompasses a plasmalogen-containing composition, a method for producing a plasmalogen-containing composition, a method for improving the stability of plasmalogen, and the like. However, the present invention is not limited to these and encompasses all of the matter disclosed in the present specification and recognized by those skilled in the art.

The plasmalogen-containing composition encompassed by the present invention comprises plasmalogen, γ-cyclodextrin, and a pH alkali adjusting agent. Below, this composition may be referred to as “the Pls-γCD-pH agent-containing composition.”

Plasmalogens usually refer to glycerophospholipids having a long-chain alkenyl group via a vinyl ether bond at position 1 (sn-1 position) of the glycerol skeleton. The formula of plasmalogens is shown below.

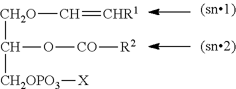

In the formula, R¹ and R² each represent an aliphatic hydrocarbon group. R¹ usually represents an aliphatic hydrocarbon group having 1 to 20 (1, 2, 3, 4, 5, 6, 7, 8, 9, 10, 11, 12, 13, 14, 15, 16, 17, 18, 19, or 20) carbon atoms, such as a dodecyl group, a tetradecyl group, a hexadecyl group, an octadecyl group, and an icosanyl group. R² usually represents an aliphatic hydrocarbon group from a fatty acid residue, such as an octadecadienoyl group, an octadecatrienoyl group, an icosatetraenoyl group, a docosatetraenoyl group, a docosapentaenoyl group, and a docosahexaenoyl group. In the formula, X represents a polar group. X preferably represents ethanol amine, choline, serine, inositol, or glycerol.

In particular, ethanol amine plasmalogen represented by the above formula in which X is ethanol amine, and choline plasmalogen represented by the above formula in which X is choline are widely present in nature; and are also preferably used as plasmalogen in the present invention.

For example, the plasmalogen used for the Pls-γCD-pH agent-containing composition may be, but are not particularly limited to, synthetic products, extracts, or the like. In particular, those extracted from tissue from a living body are preferable. Tissue from a living body refers to plasmalogen-containing tissue in a living organism. The living organisms used to extract plasmalogen include, for example, animals and microorganisms. The microorganisms are preferably anaerobic bacteria, and are particularly preferably, for example, bacteria of the family Acidaminococcaceae, which are intestinal bacteria. In terms of bacteria, the “tissue from a living body” refers to the bacterium itself. The animals are preferably birds, mammals, fish, shellfish, and the like. The mammals are preferably livestock and poultry, from the viewpoint of supply stability and safety. Examples include cattle, pigs, horses, sheep, goats, birds, and the like. In terms of mammals, examples of tissues that contain plasmalogen mainly include skin, brain, intestines, heart, reproductive organs, and the like. Plasmalogen can be extracted from these tissues. Examples of birds include chicken, domestic duck, Japanese quail, duck, green pheasant, turkey, and the like. Chicken is particularly preferable in consideration of availability, cost, palatability, etc. The bird tissue is not particularly limited, and is preferably of, for example, bird meat (in particular, bird breast meat), bird skin, internal bird organs, or the like. The shellfish is preferably, for example, a scallop. It is also possible to combine two or more different tissues of one or more different living organisms.

The plasmalogen extracted from tissue from a living body is particularly preferably plasmalogen extracted from tissue of birds. In particular, birds (birds for food) that have been conventionally edible are preferable because their safety has already been confirmed, and a stable supply thereof is easily achieved. In particular, chicken is most preferable.

The method for extracting plasmalogen from tissue from a living body is not particularly limited as long as plasmalogen can be extracted (and optionally purified). For example, extraction can be performed by a known method, or a method that is easily conceivable from a known method.

Two specific examples of methods for extracting plasmalogen from tissue from a living body are described below. However, the extraction method is not limited to these. The plasmalogen used for the Pls-γCD-pH agent-containing composition may also be a purchased commercially available product.

### EXAMPLE 1 OF METHOD FOR EXTRACTING PLASMALOGEN

Examples of the method for extracting and purifying plasmalogen include the method disclosed in PTL 3 (JP2016-210696A), and the like. More specifically, examples include a method comprising step (1) of extracting plasmalogen from tissue from a living body; step (2) of purifying plasmalogen contained in the extract (i.e., a step of removing neutral lipids and/or sphingolipids); and step (3) of hydrolyzing the extract, followed by purification (i.e., a step of hydrolyzing diacyl glycerophospholipids, followed by removal of free fatty acids and lysophospholipids). Step (1) here can be referred to as a plasmalogen extraction step, and steps (2) and (3) can be referred to as plasmalogen purification steps. Therefore, steps (2) and (3) are optional, and may be omitted. However, it is more preferable to use plasmalogen concentrated by purification; thus, the method preferably comprises at least either step (2) or (3). In particular, the method preferably comprises all of steps (1) to (3).

In this method, the solvent used when extracting plasmalogen is preferably water, an organic solvent, or a hydrous organic solvent. Examples of organic solvents include methanol, ethanol, isopropanol, hexane, and the like; and mixtures of two or more solvents selected from the group consisting of these solvents. The moisture content of hydrous organic solvents is not particularly limited. For example, a hydrous organic solvent having a moisture content of 10 to 90% (v/v) may be used. Of these, ethanol or hydrous ethanol is preferable. The tissue from a living body to be subjected to extraction may be raw, or may have been subjected to some treatment beforehand. For example, those that have been subjected to drying treatment and/or deoiling treatment may be used.

The extraction treatment method is not particularly limited. For example, the extraction treatment can be performed by an immersion method, such as cold immersion or hot immersion; a percolation method; or the like. Preferable examples include a method comprising adding 1 to 10 L, preferably 1 to 6 L, and more preferably 2 to 4 L of ethanol to 1 kg of chicken breast meat; and allowing the resulting product to stand or stirring the resulting product, at 30° C. or higher for 60 minutes or more, and preferably at 40° C. or higher, for 180 minutes or more.

The obtained organic solvent extract is preferably concentrated to dryness, and then subjected to a hydrolysis treatment step. The concentration to dryness can be conducted by a known method, for example, by using an evaporator. The thus-obtained organic solvent extract (dry solid matter of organic solvent extract) contains concentrated lipids, such as plasmalogen.

Further, the dry solid matter of organic solvent extract is preferably, for example, centrifuged with acetone to collect the precipitate, which is further centrifuged with a solvent obtained by mixing hexane and acetone (hexane-acetone mixed solvent) to collect the liquid layer. Although limited interpretation is not intended, neutral lipids can be removed by collecting the precipitate after centrifugation with acetone, and sphingolipids can be removed by collecting the liquid layer after centrifugation with a mixed solvent of hexane and acetone.

The thus-obtained liquid layer is concentrated to dryness to obtain a dry solid matter of phospholipid concentrate. The dry solid matter of phospholipid concentrate is subjected to a hydrolysis treatment step to hydrolyze diacyl glycerophospholipids to thus concentrate plasmalogen in a preferable manner.

Examples of the hydrolysis treatment include treatment with phospholipase A1 (PLA1). PLA1 specifically hydrolyzes the ester bond between the fatty acid at the sn-1 position and the glycerin skeleton in diacyl glycerophospholipids. The hydrolyzed diacyl glycerophospholipids are degraded into free fatty acids and lysophospholipids. On the other hand, plasmalogen, which has a vinyl ether bond at the sn-1 position, is not affected by PLA1. Therefore, the treatment with PLA1 can specifically degrade diacyl glycerophospholipids without degrading plasmalogen. Plasmalogen can be purified by converting diacyl glycerophospholipids coexisting with the plasmalogen into lyso forms with PLA1, and removing free fatty acids and lysophospholipids. The removal of free fatty acids and lysophospholipids can be performed, for example, by partitioning with acetone and hexane.

The origin etc. of PLA1 is not particularly limited as long as it functions as described above. Examples include PLA1 from Aspergillus oryzae. PLA1 may also be a commercially available product, and may be purchased from, for example, Mitsubishi-Chemical Foods Corporation. The amount of PLA1 used may be appropriately set according to the amount of the dry solid matter of organic solvent extract to be subjected to the hydrolysis treatment. For example, the amount may be about 0.2 to 200 units, and preferably about 2 to 200 units per 1 mg of the dry solid matter of organic solvent extract. One unit refers to an amount that modifies 1 μmol of the substrate (diacyl glycerophospholipids) per minute (1 μmol/min).

The buffer for use may also be appropriately determined according to the type of PLA1 to be used. Examples of buffers include 0.1M citrate+HCl buffer (pH 4.5), and the like. The amount of buffer used is not particularly limited as long as the enzymatic reaction is allowed to proceed. For example, the amount may be about 1 to 30 mL, and preferably about 5 to 15 mL per 1 g of the dry solid matter of organic solvent extract. PLA1 may be added after the buffer is added to the dry solid matter of organic solvent extract, and the dry solid matter of organic solvent extract is dissolved in the buffer.

The reaction conditions may also be appropriately determined. The reaction is preferably performed with stirring at 50° C. for 1 to 2 hours.

PLA1 may be subjected to deactivation treatment. For example, the deactivation treatment of PLA1 can be performed by increasing the temperature to about 70° C. after the hydrolysis reaction.

The above method can provide a treatment liquid (hydrolysis treatment liquid) in which diacyl glycerophospholipids are degraded. For example, hexane in an amount about 2 to 3 times the amount of the hydrolysis treatment liquid is added to the hydrolysis treatment liquid, and, after centrifugation, the upper layer (hexane layer) is collected, which allows enzyme buffer and enzyme proteins to be removed. (The enzyme buffer and enzyme proteins are dissolved in the lower aqueous layer and are not contained in the hexane layer.)

Plasmalogen is soluble in hexane, but is poorly soluble in acetone. Thus, by appropriately combining these solvents and water, performing partitioning, and further performing solution partitioning with water or an aqueous solution, lysophospholipids are removed, and plasmalogen can be obtained. That is, neutral lipids other than phospholipids can be removed with the use of acetone, and plasmalogen and lysophospholipids can be separated by liquid-liquid partitioning.

As is clear from the descriptions above, steps (1) to (3) above are more specifically, for example, as follows:
(1) extracting tissue from a living body with ethanol or hydrous ethanol,
(2) centrifuging the extract obtained in step (1) with acetone, collecting the precipitate, further centrifuging the precipitate with a hexane-acetone mixed solvent, and collecting the liquid layer, and
(3) treating the liquid collected in step (2) with phospholipase A1 (PLA1) (and optionally further performing partitioning with acetone and hexane to remove free fatty acids and lysophospholipids).

### EXAMPLE 2 OF METHOD FOR EXTRACTING PLASMALOGEN

Another example of the method for extracting and purifying plasmalogen includes a method comprising allowing a liquid mixture of ethanol extract concentrate of tissue from a living body and specific hydrous ethanol to stand under specific conditions. More specifically, for example, the method comprises allowing a liquid mixture comprising an ethanol extract concentrate of tissue from a living body and a 40 to 60 mass % aqueous ethanol solution in a mass ratio of 1:0.8 to 1.2 to stand at 40 to 60° C. The tissue from a living body used in this method is preferably, but not particularly limited to, tissue of chicken, and particularly preferably of chicken breast meat.

In this method, the method of ethanol extraction for tissue from a living body is not particularly limited, and may be a known method or a method that can be easily conceivable from a known method. For example, ethanol extraction can be performed by adding ethanol in an amount of about 1 to 5 times that of the tissue from a living body, based on mass, followed by stirring or allowing it to stand. The stirring or allowing it to stand may be performed with heating. The heating may be performed, for example, at about 30 to 50° C. or about 35 to 45° C. The time for stirring or allowing it to stand is not particularly limited, and may be, for example, about 0.5 to 24 hours or about 1 to 12 hours. The obtained extract may be subjected to solid-liquid separation by filtration or the like, if necessary. Further, the extraction residue may be subjected to the same operation to obtain an extract again, which may be added to the extract obtained beforehand.

When the temperature is low (in particular, in winter), a deposit may be formed during the extraction step. The temperature at which a deposit is formed is not limited, and is specifically, for example, 10° C. or lower, 9° C. or lower, 8° C. or lower, 7° C. or lower, 6° C. or lower, 5° C. or lower, 4° C. or lower, 3° C. or lower, 2° C. or lower, 1° C. or lower, or 0° C. or lower. The deposit contains phospholipids; thus, if ethanol extraction is continued while the deposit is left as is, the phospholipids contained in the deposit will not be incorporated into the ethanol extracts. For this reason, the amount of phospholipids contained in the finally obtained phospholipid concentrate may vary. Therefore, when a deposit is formed, it is preferable to first perform heating to dissolve the deposit, or perform the step at a temperature at which no deposit is formed. When heating is performed to dissolve the deposit, the heating temperature is not particularly limited as long as the deposit dissolves and as long as it does not affect the quality. For example, the temperature is about 20 to 30° C. When the ethanol extraction step is performed at a temperature at which no deposit is formed, the step can be performed at a temperature of, for example, about 20 to 30° C.

The method of concentrating the obtained ethanol extract is not particularly limited; and may be a known method, or a method that is easily conceivable from a known method. Examples include vacuum concentration, heat concentration, and the like.

The concentration is preferably performed until the water content of the obtained ethanol extract concentrate is 1 mass % or less, more preferably 0.9 mass % or less, 0.8 mass % or less, 0.7 mass % or less, 0.6 mass % or less, or 0.5 mass % or less, and still more preferably 0.4 mass % or less, 0.3 mass % or less, or 0.2 mass % or less. The water content is a value determined by the Karl Fischer method.

The ethanol content of the obtained ethanol extract concentrate is preferably 15 mass % or less, and more preferably 14 mass % or less, 13 mass % or less, 12 mass % or less, 11 mass % or less, 10 mass % or less, 9 mass % or less, or 8 mass % or less. The ethanol content is a value obtained by subtracting the water content from the loss on drying determined by a dry-heat drying method (105° C., 3 hours). For example, when the loss on drying determined by dry heating is 90 mass %, and the water content is 1 mass %, the ethanol content is 100−90−1=9 (mass %).

An ethanol extract concentrate of tissue from a living body and a 40 to 60 mass % aqueous ethanol solution are mixed in a mass ratio of 1:0.8 to 1.2. The lower limit of the mass ratio may be, for example, 1:0.85, 0.9, 0.95, or 1. The upper limit of the mass ratio may be, for example, 1:1.15, 1.1, 1.05, or 1. Further, the lower limit of the concentration of the aqueous ethanol solution used may be, for example, 41, 42, 43, 44, 45, 46, 47, 48, 49, or 50 mass %. The upper limit of the concentration of the aqueous ethanol solution used may be, for example, 59, 58, 57, 56, 55, 54, 53, 52, 51, or 50 mass %.

The thus-obtained liquid mixture is allowed to stand at 40 to 60° C. This allows the liquid mixture to separate into three layers (upper layer, middle layer, and lower layer), and plasmalogen is concentrated in the lower layer.

The lower limit of the temperature for allowing it to stand may be, for example, 41, 42, 43, 44, 45, 46, 47, 48, 49, or 50° C. The upper limit of the temperature for allowing it to stand may be, for example, 59, 58, 57, 56, 55, 54, 53, 52, 51, or 50° C. The temperature for allowing it to stand may vary within the above temperature range. However, the temperature is preferably as stable as possible. Even if the temperature changes, the range of change is preferably small (for example, within a range of about 1 to 5° C. or about 1 to 3° C.), and the rate of change is also preferably as slow as possible.

The time for allowing it to stand is not particularly limited as long as the liquid mixture separates into layers, and is preferably, for example, 1 hour or more. The time may be 2 hours or more, 3 hours or more, 4 hours or more, 5 hours or more, or 6 hours or more. The upper limit of the time for allowing it to stand is not particularly limited, and is, for example, 24 hours or less, 18 hours or less, 12 hours or less, or 10 hours or less.

As stated above, plasmalogen is concentrated in the lower layer of the liquid mixture separated into three layers. Therefore, the method for extracting plasmalogen may further comprise a step of collecting the lower layer from the liquid mixture separated into three layers after being allowed to stand.

The lower layer is collected, for example, by (i) removing the upper layer from the liquid mixture separated into three layers, and allowing the resulting product to stand at a temperature of 10° C. or lower until the lower layer is gelled, followed by removal of the middle layer, or (ii) allowing the liquid mixture separated into three layers to stand at a temperature of 10° C. or lower until the lower layer is gelled, followed by removal of the upper layer and the middle layer.

In both steps (i) and (ii), the temperature for standing is 10° C. or lower, and may be, for example, 9° C. or lower, 8° C. or lower, 7° C. or lower, 6° C. or lower, 5° C. or lower, or 4° C. or lower. Further, the time for standing is not particularly limited as long as it is within the range in which the lower layer is formed into a gel, and is, for example, 12 hours or more.

Considering the above preferable range of the ethanol content of the ethanol extract concentrate as well, the liquid mixture comprising an ethanol extract concentrate of tissue from a living body and a 40 to 60 mass % aqueous ethanol solution in a mass ratio of 1:0.8 to 1.2 can be interpreted to represent, for example, a liquid mixture comprising an ethanol extract of tissue from a living body, ethanol, and water, and having an ethanol content of 20 to 43.5 mass %. The lower limit of the ethanol content of the liquid mixture may be 21, 22, 23, 24, 25, 26, 27, 28, 29, or 30 mass %. Further, the upper limit of the ethanol content of the liquid mixture may be 43, 42, 41, 40, 39, 38, 37, 36, 35, 34, or 33 mass %. Considering the above preferable range of the water content of the ethanol extract concentrate as well, the water content of the liquid mixture may be, for example, 16 to 36.5 mass %. The lower limit of the water content of the liquid mixture may be 17, 18, 19, 20, 21, 22, 23, or 24 mass %. Further, the upper limit of the water content of the liquid mixture may be 36, 35, 34, 33, 32, 31, 30, 29, 28, 27, or 26 mass %.

For example, a plasmalogen-containing extract extracted (and optionally further purified) by the above method can be preferably used for the Pls-γCD-pH agent-containing composition.

Cyclodextrins are cyclic oligosaccharides in which several molecules of D-glucose are connected via α-1,4-glycosidic bonds to form a cyclic structure. γ-Cyclodextrin consists of linked six molecules, β-cyclodextrin consists of linked seven molecules, and γ-cyclodextrin consists of linked eight molecules. These are known compounds. γ-Cyclodextrin can also be purchased commercially and used for the Pls-γCD-pH agent-containing composition.

The pH alkali adjusting agent refers to a compound having an action of increasing an acidic pH value. It is not always necessary to adjust the pH value to alkaline, and the pH alkali adjusting agent also encompasses compounds that are capable of adjusting the pH, for example, from strongly acidic to weakly acidic or to neutral. The pH alkali adjusting agent may be known agents. In particular, pharmacologically or food-hygienically acceptable pH alkali adjusting agents are preferable. Specifically, preferable examples include sodium citrate, sodium carbonate, sodium hydrogen carbonate, sodium hydrogen phosphate, and the like. The sodium citrate may be monosodium citrate, disodium citrate, or trisodium citrate; and trisodium citrate is particularly preferable. The sodium hydrogen phosphate may be disodium hydrogen phosphate or sodium dihydrogen phosphate; and disodium hydrogen phosphate is particularly preferable. The pH alkali adjusting agents may be used alone, or in a combination of two or more.

The Pls-γCD-pH agent-containing composition may be, for example, a liquid composition or a solid composition; and is preferably a solid composition. Among solid compositions, a dry composition is preferable, and a powder composition is particularly preferable.

The Pls-γCD-pH agent-containing composition preferably has a pH of 6 to 8.

For example, when the Pls-γCD-pH agent-containing composition as a solid composition is formed into a 1 mass % aqueous suspension by being dispersed in water, the pH of the 1 mass % aqueous suspension is 6 to 8. The dispersion operation here is performed by shaking in a water bath at 55° C. for 1 hour. The pH is measured at 25° C. with a pH meter. The Pls-γCD-pH agent-containing composition, which gives a 1 mass % aqueous suspension having a pH of 6 to 8 when dispersed in water, is preferable because the stability of plasmalogen contained is high. The lower limit of the pH range may be 6.1, 6.2, 6.3, 6.4, 6.5, 6.6, 6.7, or 6.8; and the upper limit may be 7.9, 7.8, 7.7, 7.6, 7.5, 7.4, 7.3, or 7.2. As described below, the solid composition can be prepared, for example, by drying a liquid composition comprising plasmalogen, γ-cyclodextrin, and a pH alkali adjusting agent; thus, it is preferable to appropriately add a pH alkali adjusting agent at the time of preparing the liquid composition, which is a starting material of the solid composition, so that the pH is within the above pH range. In the present specification, the expression “mass %” denotes w/w % unless otherwise specified.

Further, for example, when the Pts-γCD-pH agent-containing composition is a liquid composition, the composition has a pH of preferably 6 to 8. The solvent of the liquid composition is not limited as long as plasmalogen can be dispersed, and as long as the pH of the composition becomes 6 to 8. For example, water is preferable. The liquid composition is particularly preferably an aqueous suspension. Similar to the above, the pH here is measured at 25° C. with a pH meter. The Pls-γCD-pH agent-containing liquid composition having a pH of 6 to 8 is preferable because the stability of plasmalogen contained is high. The lower limit of the pH range may be 6.1, 6.2, 6.3, 6.4, 6.5, 6.6, 6.7, or 6.8; and the upper limit may be 7.9, 7.8, 7.7, 7.6, 7.5, 7.4, 7.3, or 7.2. Since the liquid composition has a pH of 6 to 8, the solid composition described above can be preferably prepared by drying this liquid composition. In other words, the liquid composition can also be preferably used as a starting material for the solid composition.

The Pls-γCD-pH agent-containing composition can be prepared, for example, by mixing plasmalogen (which may be, for example, a plasmalogen-containing extract extracted from tissue from a living body), γ-cyclodextrin, and a pH alkali adjusting agent, and optionally a solvent (particularly preferably water). The composition obtained by this method is a liquid composition. To obtain the composition as a solid composition, for example, the obtained mixture may be dried to form a solid composition. The drying treatment may be performed by a known method. Examples include freeze-drying, spray-drying, and the like. Further, concentration treatment may be performed before the drying treatment. Examples of the concentration treatment method include vacuum concentration and the like. After the drying treatment, the obtained solid composition may be optionally crushed or the like to form a powder. When the drying treatment is performed by spray-drying, a powder composition can be directly obtained.

The amount of each component contained in the Pls-γCD-pH agent-containing composition is not particularly limited as long as the effect is obtained; i.e., as long as the stability of the contained plasmalogen is improved. For example, the composition in the form of a solid composition (in particular, a dry composition) preferably contains plasmalogen in an amount of 0.1 to 10 mass %. The lower limit of this range may be, for example, 0.2, 0.3, 0.4, 0.5, 0.6, 0.7, 0.8, 0.9, or 1 mass %. Further, the upper limit of this range may be, for example, 9.5, 9, 8.5, 8, 7.5, 7, 6.5, 6, 5.5, 5, 4.5, 4, 3.5, or 3 mass %.

Further, the Pls-γCD-pH agent-containing composition preferably comprises γ-cyclodextrin in an amount of about 10 to 100 parts by mass, and more preferably about 15 to 100 parts by mass per 1 part by mass of plasmalogen. In particular, when the composition is a solid composition (in particular, a dry composition), γ-cyclodextrin is preferably contained in an amount of about 40 to 95 mass %. The lower limit may be, for example, 45, 50, 55, 60, or 65 mass %. The upper limit may be, for example, 90, 85, 80, or 75 mass %.

The Pls-γCD-pH agent-containing composition comprises the pH alkali adjusting agent so that the pH of the composition is 6 to 8, and the amount of the pH alkali adjusting agent may be appropriately determined according to the type of the pH alkali adjusting agent to be used. In particular, when the Pls-γCD-pH agent-containing composition is a solid composition (in particular, a dry composition), and the pH alkali adjusting agent used is sodium citrate (in particular, trisodium citrate), the amount is preferably, for example, about 0.5 to 20 mass %. The lower limit of this range may be, for example, 1 or 1.5 mass %. The upper limit of this range may be, for example, 19, 18, 17, 16, 15, 14, or 13 mass %.

In addition to plasmalogen, γ-cyclodextrin, and the pH adjusting agent, the Pls-γCD-pH agent-containing composition may further comprise other components as long as the effect of the present invention is not impaired. The components may be various components known in the pharmaceutical field or the food field. For example, a pharmacologically or food-hygienically acceptable carrier may be used. More specific examples include, but are not particularly limited to, known excipients, sweeteners, binders, disintegrants, and the like. For example, when these other components are incorporated, these other components (and optionally a solvent) may be mixed as well during the preparation of the composition, in addition to plasmalogen, γ-cyclodextrin, and the pH adjusting agent.

Further, the Pls-γCD-pH agent-containing composition may be preferably used, for example, in the production of pharmaceuticals or foods.

The present invention also encompasses a method for producing a plasmalogen-containing composition, comprising step (A) of mixing at least plasmalogen, γ-cyclodextrin, a pH adjusting agent, and a solvent (preferably water) to prepare a suspension having a pH of 6 to 8. The suspension obtained in step (A) may be used as is as a plasmalogen-containing composition, or may be further dried to obtain a solid composition. It is also preferable that the method further comprises step (B) of drying the suspension obtained in step (A) to obtain a dry composition after step (A).

The present invention also encompasses a method for improving the stability of plasmalogen, comprising step (A) of mixing at least plasmalogen, γ-cyclodextrin, a pH adjusting agent, and a solvent (preferably water) to prepare a suspension having a pH of 6 to 8. It is also preferable that the method further comprises step (B) of drying the suspension obtained in step (A) to obtain a dry composition after step (A).

For these methods, the above description for the Pls-γCD-pH agent-containing composition can be preferably applied as is.

In the present specification, the terms “comprising” and “containing” also includes the meanings of “consisting essentially of” and “consisting of.” Further, the present invention includes all possible combinations of the constituent requirements described in the present specification.

The various characteristics described above in terms of each embodiment of the present invention (e.g., properties, structures, functions) may be combined in any way in specifying the subject matter encompassed by the present invention. More specifically, the present invention encompasses all of the subject matter of all possible combinations of each characteristic described in the present specification.

### Examples

The present invention is described in more detail below. However, the present invention is not limited to the following Examples. For the starting material for extracting plasmalogen, freeze-dried chicken breast meat (FD chicken breast meat) was used. The expression “%” denotes mass (w/w) % unless otherwise specified.

### Ethanol Extraction

FD chicken breast meat (42 kg) was placed in an extraction tank. Then, 99% ethanol in an amount 4 times (w/v; 168 L) that of the FD chicken breast meat was placed in the extraction tank. After the tank was purged with nitrogen, the mixture was heated with stirring; and, when the temperature reached 40° C., the mixture was allowed to stand for 90 minutes. After filtration through a 30-mesh filter, the extract was collected in a drum. The first extraction residue was placed in the extraction tank, and 99% ethanol in an amount 2.5 times (w/v; 105 L) that of the FD chicken breast meat was placed in the extraction tank. After the tank was purged with nitrogen, the mixture was heated with stirring; and, when the temperature reached 40° C., the mixture was allowed to stand for 90 minutes. After filtration through a 30-mesh filter, the extract was collected in a drum. The extract was suction-filtered with 10S filter paper. The obtained liquid was used as an ethanol extract.

Concentration of Ethanol Extract under Reduced Pressure

The ethanol extract was concentrated under reduced pressure at an internal temperature of 40° C. or lower to about a volume that could be entirely contained in a 50-L vat (about 8 kg). The resulting primary concentrated liquid was placed in a 50-L vat, and the pressure was reduced at an external temperature of 50 to 60° C. After the obtained concentrated liquid was filtered through a 30-mesh filter, the weight was measured. The weight was 5.12 kg. This concentrated liquid for use as an ethanol extract concentrate was stored at 4° C. until use.

The loss on drying of the ethanol extract concentrate was determined by a dry-heat drying method (105° C., 3 hours), while the water content was determined by the Karl Fischer method. The ethanol concentration was calculated by the subtraction method. As a result, the loss on drying was 8.05%, the water content was 0.15%, and the ethanol was 7.9%.

### Study on Mixing of Ethanol Extract Concentrate and Aqueous Ethanol Solution

### Study on Separation 1

To 15 g of the ethanol extract concentrate, an equivalent amount of 50, 60, 70, or 80% aqueous ethanol solution (w/w) was added, and the mixture was stirred at room temperature. After the mixture was allowed to stand at room temperature for 3 hours, the state of separation was confirmed.

### Study on Separation 2

To 10 g of the ethanol extract concentrate, an equivalent amount of 25, 50, or 75% aqueous ethanol solution (w/w) was added, and the mixture was heated with stirring until the temperature reached 50° C. After the mixture was allowed to stand at 50° C. for 3 hours, the state of separation was confirmed. TLC Analysis

The components contained in the separated layers in each study were confirmed by thin-layer chromatography (TLC). More specifically, each layer separated in Study 1 and Study 2 was developed with a mobile phase (chloroform/methanol/water=65/25/4) using a thin layer made of a thin-layer plate (silica gel), and neutral lipids were separated from phospholipids. For the detection solution, 10% sulfuric acid was used.

In Study 1, when the 50% aqueous ethanol solution was mixed in the equivalent amount, the mixture became an emulsion, and separation could not be observed. When the 60, 70, or 80% aqueous ethanol solution was mixed in the equivalent amount, separation into two layers was observed. However, the lipid distribution of each layer in TLC confirmed insufficient separation since neutral lipids and phospholipids were present in both of these layers. These results clarified that the separation under room temperature conditions was insufficient.

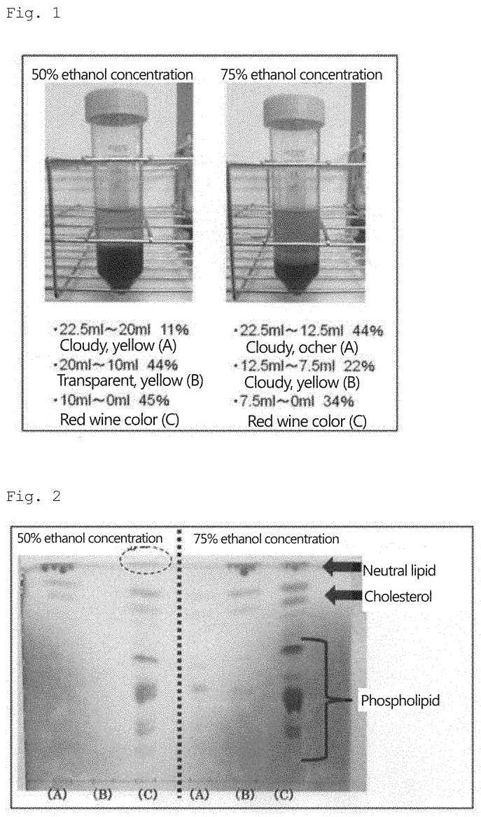

In Study 2, when the 25% aqueous ethanol solution was mixed in the equivalent amount, the mixture became an emulsion, and separation could not be observed. However, when the 50 or 75% aqueous ethanol solution was mixed in the equivalent amount, separation into three layers was observed (FIG. 1). TLC analysis revealed that when the 75% ethanol was mixed in the equivalent amount, the lower-layer fraction containing the largest amount of phospholipids also contained large amounts of neutral lipids and cholesterol. In contrast, when the 50% aqueous ethanol solution was mixed in the equivalent amount, it was confirmed that neutral lipids were mainly present in the upper layer while phospholipids were mainly present in the lower layer (FIG. 2). In FIG. 2, (A) represents the upper layer, (B) represents the middle layer, and (C) represents the lower layer.

The above results revealed that when a 50% aqueous ethanol solution was mixed in an equivalent amount, and the mixture was allowed to stand at 50° C., a phospholipid concentrate was efficiently obtained from the ethanol extract concentrate of bird breast meat even without centrifugation.

Therefore, as described above, the equivalent amount (w/w) of a 50% aqueous ethanol solution was added to the ethanol extract concentrate, and the mixture was heated to 50° C. with stirring. After the resulting product was allowed to stand at 50° C. for 3 hours, the lower layer was collected from the separated three layers and used in the following studies as a plasmalogen-containing chicken breast meat extract. Below, the “chicken breast meat extract” refers to this plasmalogen-containing chicken breast meat extract.

### Study on Chicken Breast Meat Extract

Thirty milliliters of chloroform/methanol (2:1, v/v) was added to 0.2 g of the chicken breast meat extract to extract lipids; and 7.5 mL of 0.9% potassium chloride solution was added thereto, followed by shaking. Subsequently, centrifugation was performed to separate the liquid into two layers. Thereafter, the lower layer was collected, and lipids alone (about 0.13 g) were collected by distilling off the solvent. The preparation of the chicken breast meat extract was repeated multiple times, and the amount of lipids contained was examined, which revealed that about 60 to 70 mass % of the chicken breast meat extract was lipids.

Thereafter, fractionation was performed on a silica gel column, and only a phospholipid-containing fraction was isolated and subjected to HPLC separation in accordance with a known method (see Non-patent Literature (NPL) 1: Animal Science Journal, 85 (2), 153-161, 2014) to quantify Pls. The details are as follows. The extracted lipids (20 mg) were dissolved in a small amount of chloroform, and 30 mL of chloroform was passed through a silica gel column to allow a neutral lipid fraction to be eluted. Thereafter, chloroform/methanol (2:1 v/v) and 30 mL of methanol were passed through the silica gel column to isolate a polar lipid fraction (phospholipid-containing fraction). Further, the phospholipid-containing fraction was subjected to HPLC analysis under the following conditions, and the amount of plasmalogen was measured.

### HPLC Analysis

Device: LC-20AD (Shimadzu Corporation, Kyoto), mobile phase: solution A (hexane/2-propanol:acetic acid (82:17:1, v/v/v), solution B (2-propanol/water/acetic acid (85:14:1, v/v/v)+0.2% triethylamine, gradient conditions (solution B %): 0-1 min (0-5%), 1-25 min (5-40%), and 25-28 min (40-0%), column: LiChrospher 100-Diol (250 mm×4 mm, particle size: 5 μm; Merck Millipore), flow rate: 1 mL/min, sample volume: 10 μg, column temperature: 50° C., detector: ELSD-LTII (50° C., 350 kPa; Shimadzu Corporation)

The plasmalogen quantification was performed based on the peak area of plasmalogen detected in HPLC chromatogram. More specifically, the peak position of plasmalogen was confirmed beforehand by analyzing a standard substance. The quantification based on the peak area was performed by analyzing a standard substance with a known concentration, and creating a standard curve from the obtained area value and the standard substance concentration. For the standard substances of plasmalogen, C18(Plasm)-18:1 PE and C18(Plasm)-18:1 PC (Avanti Polar Lipids) were used.

About 0.025 g of plasmalogen was contained in 0.2 g of the chicken breast meat extract. The quantitative analysis of plasmalogen was repeated multiple times, which revealed that about 10 to 15 mass % of the chicken breast meat extract was plasmalogen.

### Study 1 on Method for Improving Plasmalogen Stability

To find a substance that improves the stability of plasmalogen, cyclodextrins were first analyzed. Cyclodextrins have a cyclic structure in which several glucoses are linked by α-1,4 bonds. Due to this characteristic structure, the exterior of the ring is hydrophilic while the internal cavity is lipophilic, and cyclodextrins in water can thus encapsulate fat-soluble substances in their cavity. This phenomenon is generally referred to as inclusion, and cyclodextrins are expected to improve the stability of the included fat-soluble substances. Accordingly, cyclodextrins are used to stabilize functional food materials with an antioxidant ability, such as coenzyme Q10 and α-lipoic acid.

Cyclodextrins may be referred to as “CDs.” Further, α-cyclodextrin and γ-cyclodextrin may be referred to as “αCD” and “γCD,” respectively. Additionally, plasmalogen may be referred to as “Pls.”

### Stabilization Study 1

Five hundred grams of αCD or γCD (CycloChem Co., Ltd.) and 1500 g of deionized water were added to 175 g of the chicken breast meat extract, and the mixture was stirred using a homogenizer (6000 rpm, 20 minutes, room temperature). After stirring, the sample was frozen, and after freeze-drying, the sample was crushed, thus preparing a powder.

The chicken breast meat extract and CD were individually heated at 105° C. for 1 hour to determine the loss on drying. Then, the value obtained by subtracting the loss on drying from the original amount was defined as the solids content. Since the chicken breast meat extract mainly consists of water, ethanol, and lipids, the solids content is almost equal to the amount of lipids contained. Since CDs contain a slight amount of water, the solids content represents an amount of CDs in the state in which water has been removed. When the plasmalogen-containing composition is prepared as a dry composition, as in this study, the solids content reflects the amounts of lipids and CDs contained in the dry composition.

Table 1 shows the amounts, the solids contents, and the solids percentages (mass %) of the chicken breast meat extract and CD (α or γ). Table 1 also shows the amount, the solids content, and the solids percentage (mass %) of the Pls contained.

TABLE 1

| | | Solids | Solids |
|---|---|---|---|
| | Amount (g) | content (g) | percentage (%) |
| Chicken breast meat extract | 175.0 | 120.2 | 20.9 |
| CD(α, γ) | 500.0 | 455.0 | 79.1 |
| Total | 675.0 | 575.2 | 100.0 |
| Plasmalogen | 21.88 | 21.88 | 3.8 |

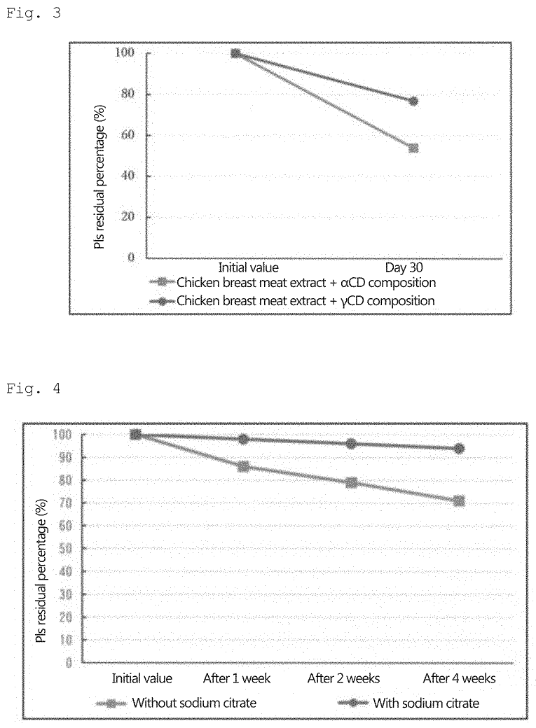

The obtained powder was subjected to an accelerated test at 40° C., and the amount of Pls was measured on day 30. Specifically, the measurement was performed as follows. Twenty-four milliliters of 0.1M phosphate buffer (pH 7.0) was added to 0.1 g of the obtained powder, and the mixture was shaken at 55° C. for 30 minutes. Thereafter, 32 mL of methanol was added thereto, and the mixture was shaken for another 15 minutes. Subsequently, 64 mL of chloroform was added thereto to extract lipids. The obtained lipids were separated and analyzed on a silica gel column, followed by HPLC in the same manner as above to quantify Pls. Table 2 shows the results. In the results, the Pls stability was evaluated based on the ratio of the Pls quantitative value after the completion of each storage period to the Pls quantitative value in the powder immediately after the powder preparation (initial value) (Pls residual percentage %). Table 3 shows the results. FIG. 3 is a graph of Table 3.

TABLE 2

| | Pls content (g) | |
|---|---|---|
| | Initial | |
| | value | Day 30 |
| Chicken breast meat extract + αCD composition | 3.12 | 1.69 |
| Chicken breast meat extract + γCD composition | 3.21 | 2.50 |

TABLE 3

Pls residual percentage when
the initial value was 100 (%)

| | Initial | |
|---|---|---|
| | value | Day 30 |
| Chicken breast meat extract + | 100 | 54 |
| αCD composition | | |
| Chicken breast meat extract + | 100 | 77 |
| γCD composition | | |

The residual percentage on day 30 at 40° C. was 54 mass % when αCD was used, and 77 mass % when γCD was used. Both CDs used in this study resulted in a decrease in the Pls residual percentage on day 30 at 40° C., and the decrease was significant in αCD. These results suggested that γCD was a component suitable for stabilization of Pis, although the effect was not particularly considerable.

### Study 2 on Method for Improving Plasmalogen Stability

As stated above, although γCD contributed to the stabilization of Pls, its effect was not sufficient. Therefore, further study was made on a method for improving the stability of Pls. More specifically, study was made by further incorporating various components to confirm whether the addition of other components in addition to γCD could further improve the stability of Pls. The results indicated the possibility of further improving the stability of Pls by further incorporating sodium citrate.

Specifically, the study was conducted as follows. Trisodium citrate was used as the sodium citrate. In a sample that did not contain sodium citrate (control group), 30.8 g of γCD (CycloChem Co., Ltd.) and an appropriate amount of deionized water were added to 2.43 g of the chicken breast meat extract, and the mixture was stirred with a stirrer, thus preparing a suspension. The pH of the obtained suspension measured at 25° C. with a pH meter was 5.0. Thereafter, the sample was frozen and freeze-dried, followed by crushing to prepare a powder. In a sample that contained sodium citrate, 0.55 g of sodium citrate was used in addition to the starting materials used in the control group, and the mixture was stirred to prepare a suspension in the same manner. The pH of the obtained suspension measured at 25° C. with a pH meter was 7.0. Thereafter, this suspension was used to prepare a powder in the same manner as in the control group. Each of the obtained powders was subjected to an accelerated test at 60° C., and the amount of Pls was measured 1 week, 2 weeks, and 4 weeks after the start of storage in the same manner as above. Table 4 shows the composition of the powder that contained sodium citrate. (Table 4 shows that 0.30 g of Pls was contained in 2.4 g of the chicken breast meat extract.)

TABLE 4

| | | Solids | Solids |
|---|---|---|---|
| | Amount (g) | content (g) | percentage (%) |
| Chicken breast meat extract | 2.4 | 1.7 | 5.4 |
| γCD | 30.8 | 28.6 | 92.8 |
| Trisodium citrate | 0.6 | 0.6 | 1.8 |
| Total | 33.8 | 30.9 | 100.0 |
| Plasmalogen | 0.30 | 0.30 | 1.0 |

The powder prepared by adding γCD and sodium citrate to the chicken breast meat extract was dispersed in water again to measure the pH. Specifically, 10 mL of ion-exchanged water was added to 0.1 g of the powder and dispersed by shaking in a water bath at 55° C. for 1 hour. The pH of the obtained suspension measured at 25° C. with a pH meter was 7.08.

Table 5 shows the Pls residual percentage after the completion of each storage period. FIG. 4 is a graph of Table 5.

TABLE 5

Pls residual percentage when
the initial value was 100 (%)

| | Initial | After | After | After |
|---|---|---|---|---|
| | value | 1 week | 2 weeks | 4 weeks |
| Without sodium citrate | 100 | 86 | 79 | 71 |
| With sodium citrate | 100 | 98 | 96 | 94 |

The control group (without sodium citrate) showed a decrease in the Pls residual percentage from 1 week after the start of storage, and the Pls residual percentage decreased to 71% after 4 weeks. On the other hand, the powder that contained sodium citrate showed almost no decrease in the Pls residual percentage, compared to the control group, and the Pls residual percentage was as high as 94% after 4 weeks. These results revealed that the stability of Pls further improves in the powder that contained not only γCD, but also sodium citrate.

### Study 3 on Method for Improving Plasmalogen Stability

In the above studies, the powder was prepared by crushing after freeze-drying. Here, a study was conducted to confirm whether the same effect would be obtained with a powder prepared by spray-drying, which more easily enables mass preparation.

The details are as follows: 873.8 g of γCD (CycloChem Co., Ltd.), 145 g of sodium citrate, and 1800 g of deionized water were added to 323.6 g of the chicken breast meat extract, and the mixture was stirred using a homogenizer (3500 rpm, 20 minutes, room temperature) to obtain a suspension. The pH of the obtained suspension measured at 25° C. with a pH meter was 6.8. The suspension was dried using a spray dryer to obtain a powder (Table 6). The obtained powder was subjected to an accelerated test at 60° C., and the amount of Pls was measured 1 week and 2 weeks after the start of storage in the same manner as above. Table 6 shows the formulation of the powder that contained sodium citrate. (Table 6 shows that 40.5 g of Pls was contained in 323.6 g of the chicken breast meat extract used.)

TABLE 6

| | | Solids | Solids |
|---|---|---|---|
| | Amount (g) | content (g) | percentage (%) |
| Chicken breast meat extract | 323.6 | 210.3 | 18.3 |
| γCD | 873.8 | 795.1 | 69.0 |
| Trisodium citrate | 145.0 | 145.0 | 12.7 |
| Total | 1342.4 | 1150.5 | 100.0 |
| Plasmalogen | 40.5 | 40.5 | 3.5 |

The powder was dispersed in water again to measure the pH. Specifically, 10 mL of ion-exchanged water was added to 0.1 g of the powder and dispersed by shaking in a water bath at 55° C. for 1 hour. The pH of the obtained suspension measured at 25° C. with a pH meter was 7.16.

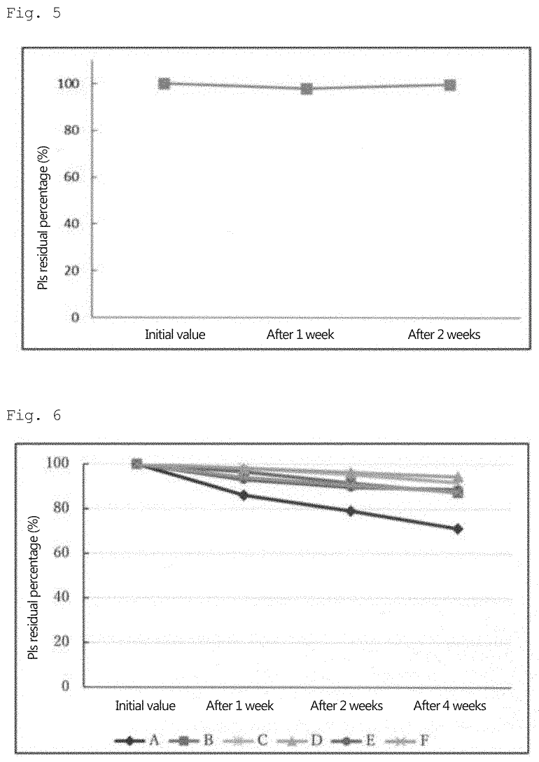

FIG. 5 shows the Pls residual percentage after the completion of each storage period. No decrease in the Pls residual percentage was observed until 2 weeks later, and the powder prepared by spray-drying also showed excellent stability equal to or higher than that of the powder prepared by freeze-drying.

### Study 4 on Method for Improving Plasmalogen Stability

The results of the above studies suggested that it may be important that the obtained powder composition when dispersed in water had a pH close to neutral for plasmalogen to stably present in the composition. Therefore, study was conducted to confirm whether the stability of plasmalogen would also improve even with the use of a pH alkali adjusting agent other than sodium citrate.

The chicken breast meat extract, γCD, water, and various pH alkali adjusting agents were stirred in the same manner as above to prepare suspensions, and each suspension was freeze-dried to prepare powders. At this time, the composition of each suspension was adjusted so that the pH of the suspension before freeze-drying was neutral (near 7: about 6.5 to 7.5) (25° C., measured with a pH meter). Table 7 below shows the compositions. Each of the obtained powders was subjected to an accelerated test at 60° C. in the same manner as above, and the amount of Pls was measured 1 week, 2 weeks, and 4 weeks after the start of storage in the same manner as above. Table 7 shows the compositions of the powders that contained various pH alkali adjusting agents. (Table 7 shows that 0.30 g of Pls was contained in 2.4 g of the chicken breast meat extract used.)

TABLE 7

| | Chicken | | PH alkali |
|---|---|---|---|
| | breast meat | | adjusting |
| Type of pH alkali adjusting agent | extract (g) | γCD (g) | agent (mg) |
| A: Control | 2.43 | 30.8 | 0 |
| B: Sodium hydrogen carbonate | 2.43 | 30.8 | 38.2 |
| C: Sodium carbonate | 2.43 | 30.8 | 20 |
| D: Trisodium citrate | 2.43 | 30.8 | 550 |
| E: Disodium hydrogen phosphate | 2.43 | 30.8 | 250 |
| F: Disodium hydrogen phosphate | 2.43 | 30.8 | 150 |

Table 8 shows the Pls residual percentages after the completion of each storage period. FIG. 6 is a graph based on Table 8.

TABLE 8

Pls residual percentage when
the initial value was 100 (%)

| | Initial | After | After | After |
|---|---|---|---|---|
| | value | 1 week | 2 weeks | 4 weeks |
| A | 100 | 86.1 | 79.1 | 71.3 |
| B | 100 | 96.6 | 91.6 | 87.4 |
| C | 100 | 98.2 | 95.5 | 91.9 |
| D | 100 | 98.2 | 96.4 | 94.5 |
| E | 100 | 93.1 | 89.7 | 88.8 |
| F | 100 | 94 | 90.6 | 87.8 |

Each powder was dispersed in water again to measure the pH. Specifically, 10 mL of ion-exchanged water was added to 0.1 g of the powder and dispersed by shaking in a water bath at 55° C. for 1 hour, and the pH of the obtained suspension was measured at 25° C. with a pH meter. Table 9 shows the results.

TABLE 9

| | pH | | | |
|---|---|---|---|---|
| | Initial | After | After | After |
| | value | 1 week | 2 weeks | 4 weeks |
| A: Control | 5.09 | 5.31 | 5.38 | 5.54 |
| B: Sodium hydrogen carbonate | 7.34 | 7.13 | 7.15 | 6.69 |
| C: Sodium carbonate | 7.11 | 7.17 | 7.02 | 7.09 |
| D: Trisodium citrate | 6.96 | 7.01 | 7.01 | 6.86 |
| E: Disodium hydrogen phosphate | 7.56 | 7.52 | 7.54 | 7.4 |
| F: Disodium hydrogen phosphate | 7.32 | 7.29 | 7.35 | 7.2 |

These results revealed that the stability of plasmalogen further improved by adjusting the pH to near neutral using a pH alkali adjusting agent in addition to γCD. Among the pH alkali adjusting agents, the results revealed that sodium citrate exhibited a particularly excellent effect in terms of improving the stability of plasmalogen.

1. A plasmalogen-containing solid composition comprising
plasmalogen,
γ-cyclodextrin, and
a pH alkali adjusting agent, and
having a pH of 6 to 8 when formed into a 1 mass % aqueous suspension.

2. A plasmalogen-containing solid composition according to claim 1, comprising
plasmalogen,
γ-cyclodextrin, and
at least one member selected from the group consisting of sodium citrate, sodium carbonate, sodium hydrogen carbonate, and sodium hydrogen phosphate, and
having a pH of 6 to 8 when formed into a 1 mass % aqueous suspension.

3. The composition according to claim 1, which is a dry composition.

4. The composition according to claim 1, which is a powder composition.

5. The composition according to claim 1, wherein the composition comprises plasmalogen in an amount of 0.1 to 10 mass %.

6. A suspension comprising
plasmalogen,
γ-cyclodextrin, and
a pH alkali adjusting agent, and
having a pH of 6 to 8.

7. A suspension according to claim 6, comprising
plasmalogen,
γ-cyclodextrin, and
at least one member selected from the group consisting of sodium citrate, sodium carbonate, sodium hydrogen carbonate, and sodium hydrogen phosphate, and
having a pH of 6 to 8.

8. The suspension according to claim 6, wherein the solvent is water.

9. A method for producing the plasmalogen-containing composition according to claim 1, comprising
step (A) of mixing at least plasmalogen, γ-cyclodextrin, a pH adjusting agent, and water to prepare a suspension having a pH of 6 to 8.

10. The method according to claim 9, further comprising
step (B) of drying the suspension obtained in step (A) to obtain a dry composition.

11. A method for improving the stability of plasmalogen, comprising
step (A) of mixing at least plasmalogen, γ-cyclodextrin, a pH adjusting agent, and water to prepare a suspension having a pH of 6 to 8.

12. The method according to claim 11, further comprising
step (B) of drying the suspension obtained in step (A) to obtain a dry composition.

13. The composition according to claim 2, which is a dry composition.

14. The composition according to claim 2, which is a powder composition.

15. The composition according to claim 2, wherein the composition comprises plasmalogen in an amount of 0.1 to 10 mass %.
